(19) United States
(12) Patent Application Publication
(10) Pub. No.: US 2012/0179138 A1
(43) Pub. Date: Jul. 12, 2012

(54) DEVICE AND METHOD FOR PATIENT ENTERAL HYDRATION

(76) Inventors: Jeffrey Lee Wheeler, Castle Rock, CO (US); Kari Nicole Whitaker Wheeler, Castle Rock, CO (US)

(21) Appl. No.: 13/347,274

(22) Filed: Jan. 10, 2012

Related U.S. Application Data
(60) Provisional application No. 61/431,309, filed on Jan. 10, 2011.

(51) Int. Cl. A61M 31/00

(52) U.S. Cl. 604/516; 604/250

(57) ABSTRACT

A device and method for enterally hydrating a patient, such as a paralyzed or limited upper body mobility patient with a functional gastrointestinal system who is nonetheless incapable or not fully capable of drinking unassisted. Here fluids are provided by a suspended semi-flexible container with graduation markings to allow for convenient estimation of fluid use, an upper open and closeable cap to allow the container to be refilled, and a lower fluid conducting tube that terminates in a mouthpiece, which contains a fluid valve formed from at least one slit in an elastic material. Patient mouth force on the valve causes the slit to enlarge, thus enabling fluid to flow into the patient.

### CROSS REFERENCE TO RELATED APPLICATIONS

This invention claims the priority benefit of U.S. provisional patent application 61/431,309 entitled “HYDRATION APPARATUS”, filed Jan. 10, 2011, inventor Jeffrey Lee Wheeler; the contents of which are incorporated herein by reference.

### BACKGROUND OF THE INVENTION

1. Field of the Invention

This invention is in the field of therapeutic devices for enterally (e.g. by mouth) hydrating patients.

2. Description of the Related Art

Ensuring that a patient is adequately hydrated is one of the most fundamental levels of medical care. Absent adequate hydration, blood pressure can fall dangerously low, blood may fail to adequately circulate, and organs may begin shutting down.

As a result, there has been a substantial amount of prior art interest in ensuring that patients are adequately hydrated. At present, the first line of hydration often begins with placing an IV line for direct parenteral administration of fluid directly into the patient's veins, it is often not ideal to keep this up for long. IV administration requires sterile fluids and IV lines, can expose the patient to some risk of infection, and other complications. If the patient is subsequently able to drink fluids without assistance, IV fluid administration will often be stopped and the patient directly “stepped down” to self enteral administration (e.g. by mouth). This self enteral administration can be as simple as providing the patient with a cup often a pitcher of fluid, along with instructions as to how much to drink.

Some patients, however, remain unable to drink fluids without assistance, often for some period of time. These “unable to drink” patients generally fall into two categories. One category may suffer from severe sickness that hampers the enteral fluid administration route, and/or be so incapacitated as to be unable to swallow (i.e. many intensive care patients). A second category may have an adequately functioning enteral system (i.e. able to swallow, stomach can handle fluids, intestine can absorb fluids and so on), yet be unable to use a cup without assistant. This can range from individuals with both upper arms in a cast, to other upper extremity injuries, arm amputations, partial or total paralysis, and the like.

At present patients who are at least able to sip and swallow and otherwise adequately handle fluids by mouth are often stepped down from IV administration, and their fluid needs are instead handled by assisted enteral administration. This assistance is often done by having a nurse or family member hold the cup up to the patient's lips, and allow them to sip and swallow. However this assisted enteral fluid administration route is quite time consuming, and whenever possible, the preferred option is to step the assisted enteral administration patient down to self enteral administration as soon as feasible.

Because of the high amounts of labor required for assisted enteral administration, other workers have suggested methods to automate or semi-automate the process.

For example, Deane in U.S. Pat. No. 2,756,740 proposed a drinking device for hospital patients that provided a push button valve to enable a patient to receive fluids from an overhead suspended container.

Similarly, Metz in U.S. Pat. No. 2,969,064 proposed a drinking tube device with a clamp-like mouthpiece with a unique internal structure that rotated and rocked to dispense fluid when activated by biting.

Similarly Edstrom in U.S. Pat. No, 5,484,405 proposed a drinking device for handicapped persons that had a semi-permanently positioned mouthpiece that was held into position by a stiffener rod, wire, or other material that was affixed to the tube that transmits fluids from the fluid reservoir to the device's mouthpiece.

More complex electronic devices were taught by Turner in U.S. Pat. No. 4,966,580. Here a nipple held on the patient's tongue has a sensor that, in response to the patient's sucking the nipple or pressing the nipple, sends an electronic signal to a peristaltic pump to pump fluids into the patient's mouth.

Despite these and other prior art proposals, none of these devices has apparently been successful enough to catch the attention of the medical community. Even recent review articles, such as “A guide to enteral access procedures and enteral nutrition” O'Keefe, s. J. D. Nat. Rev. Gastroenterol. Hepatol. 6, 207-215 (2009) show that clinician's primarily view enteral administration as either traditional utensils, more heroic interventions such as nasogastric feeding tubes, and not much else. Thus improved methods of enteral fluid administration that fall somewhere in-between a traditional cup and a nasogastric tube would be medically useful.

### BRIEF SUMMARY OF THE INVENTION

The invention is based, in part, on the insight that at least some of the problems with prior art enteral fluid administration schemes is caused by the fact that the earlier devices had generally not adequately solved the fluid valve problem in the patient's mouthpiece. Some sort of well functioning mouthpiece fluid valve is needed in order to regulate fluid flow, delivering fluid when the patient wants it, and promptly shutting off when the patient is done drinking.

Although the previously discussed complex fluid valves from prior art medically related designs have generally proven not to be successful, more recently, the sporting equipment world has come up with a number of both simple, low cost, yet highly effective fluid valves to enable athletes to drink without using their hands or arms while running, cycling, hiking, and the like.

For example, Fawcett, In U.S. Pat. No. 5,085,349 disclosed a “resilient valve and dispensing system for bicyclists” that is both simple and robust. Further, Cascade Designs Inc., of South Seattle, Wash. produces a platypus mouthpiece that is also simple and robust (see, for example, Getzewich “Bite Valve for Personal Hydration Devices and a Method for Making the Same”, U.S. publication number 2002/0011583; and Lerner, “Dispensing Valve for a Flexible Liquid Container”, U.S. Pat. No. 5,730,336. Other sporting equipment organizations, such as Camelbak Products, LLC, Petaluma, Calif. also produce simple and robust fluid valves which may potentially be useful for these purposes as well.

Thus the invention is based, in part, on the insight that the way to move forward in the field of patient enteral hydration techniques is to develop a more modern patient self-administered enteral device based various state-of-the art methods in plastics and fluid valve technology.

The invention is also based, in part, on the insight that what is needed is a new type of hydration device that configured somewhere between an IV bag on the one hand, and a sports/fitness like personal hydration system (e.g. CamelBak™, Platypus™ etc).

Thus in one embodiment, the invention may be a device and method for enterally hydrating a patient, such as a paralyzed or limited upper body mobility patient with a functional gastrointestinal system who is nonetheless incapable or not fully capable of drinking unassisted. Here fluids are provided by a suspended flexible or semi-flexible container with graduation markings to allow for convenient estimation of fluid use, an upper open and closeable cap to allow the container to be refilled, and a lower fluid conducting tube that terminates in a mouthpiece, which contains a fluid valve formed from at least one slit in an elastic material. Patient mouth force on the valve causes the slit to enlarge, thus enabling fluid to flow into the patient.

### BRIEF DESCRIPTION OF THE DRAWINGS

FIG. 1 shows a flow chart of the hydration decision tree and hydration options generally used for patient hydration management.

FIG. 2 shows an overview of the patient enteral hydration device.

FIG. 3A shows a close up of one embodiment of the devices' snap-on cap.

FIG. 3B shows a close up of an alternative embodiment of the devices' snap-on cap.

FIG. 4A shows a close up of the complementary edge of the device's snap-on cap.

FIG. 4B shows a cross sectional view of the snap-on cap in the shut position.

FIG. 5 shows a view of the device's fluid reservoir and fenestrated opening.

FIG. 6A shows an overall view of the device's mouthpiece.

FIG. 6B shows a close up view of the device's valve.

FIG. 7 shows an example of the device in use hydrating a patient.

FIG. 8 shows a flow chart of the hydration decision tree and hydration options generally used for patient hydration management, when using the invention.

FIG. 9 shows an alternate embodiment of the invention's fluid container.

### DETAILED DESCRIPTION OF THE INVENTION

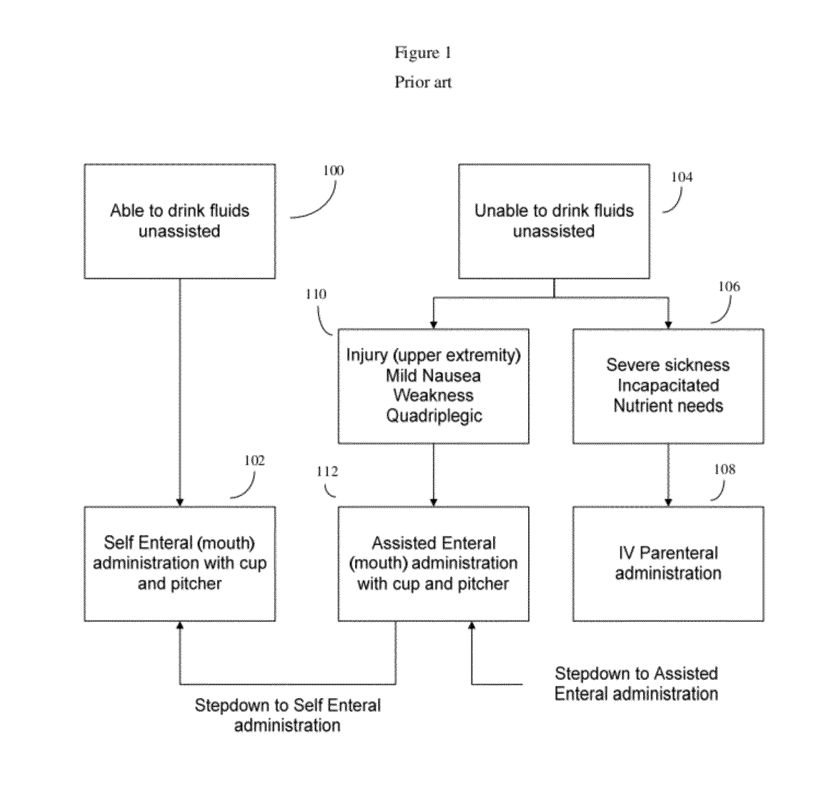

FIG. 1 shows a flow chart of the hydration decision tree and hydration options generally used for patient hydration management. At present, if a patient is able to drink fluids without outside assistance (100), then absent other reasons to give the patient an IV, the patient will normally obtain most fluids by simply drinking out of a cup (102), with the water or other beverage refreshed by a pitcher or other bottle as needed. A fair number of patients, particularly patients in clinical settings such as hospitals, rehabilitation facilities, hospices and the like are unable to drink unassisted however (104). Some of these patients may be so severely sick (i.e. major gastrointestinal issues, in intensive care, need IV administration for high dose chemotherapy, and the like (106) that they must be hydrated by IV (108).

There is also an intermediate range of patients (110) that have adequately functioning gastrointestinal systems (here adequate means capable of absorbing sufficient fluid to adequately hydrate the patient) who have other problems that prevent them from adequately self hydrating. They may be too weak, suffer from paralysis or other loss of upper body mobility (here simply having both arms in casts might be sufficient), or other problem that prevents them from self administering fluids. At present, prior inventions in the area notwithstanding, the solution is to generally throw a lot of resources at the problem by designating a nurse, nurse assistant, or family member to stand by and administer fluids (112). This is both costly, and can create gaps where the patient is awake but uncomfortable due to thirst.

By contrast, through use of the invention's device and methods, compliance with the prescribed therapeutic hydration regimen becomes both logistically similar and more reliable.

Whenever possible, clinicians attempt to step the patient down to normal hydration methods as quickly as possible. Sometimes patents can be stepped down from IV administration (108) directly to self administration with a cup (102) directly. At other times, patients will initially start with assisted administration with, for example a cup (112) and step down to self administration with a cup (102) when ready. At still other times, a patient may need to step down twice—once from IV administration (108) assisted cup administration (112), and then again to the normal self cup administration (102). The invention is based in part on the insight that there remains room for improvement at the assisted cup administration stage (112).

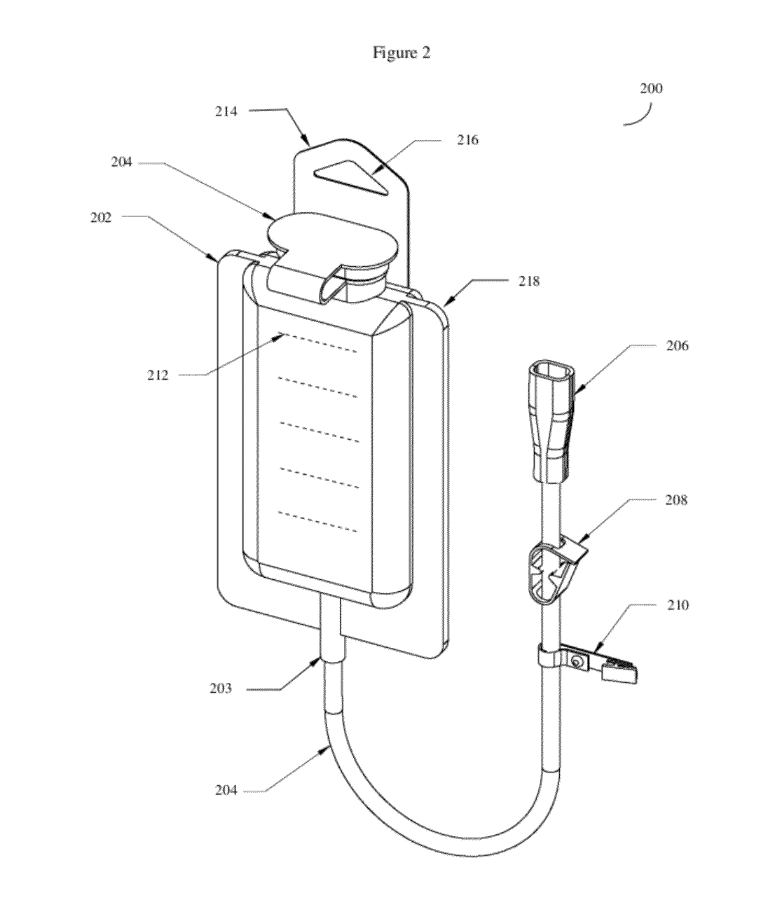

FIG. 2 shows an overview of the patient enteral hydration device (200).

In particular, in one embodiment, the invention may be a both a device and a method of enterally hydrating a patient, such as a patient in need of assisted enteral fluid administration (112).

Here, the device and method will often work by providing a substantially transparent fluid container (often plastic) (202) which will usually have several soft deformable sides (such the flexible soft and deformable sides of an IV bag), but which in some embodiments may also have at least one substantially non-deformable side. In other embodiments all sides may be flexible and deformable. The top side of this fluid container may be at least in part be substantially rigid, often with a one-piece rigid or semi-rigid press-clamp cap (204) configured to either open to admit fluids into the container, or to securely snap shut.

The back side of the fluid container will often be mounted on (or by itself) a back support (214), optionally (218). This back support, which optionally will be substantially flat, will generally extend above the level of the cap, and will often have a fenestrated opening (e.g. a hole in the back support material 216) that is also above the level of the cap (204). The hole or fenestration in the back support (216) will usually also extend above the cap, and this hole will usually be disposed to enable the container to be suspended from an IV pole or other support structure;

The lower portion of the fluid container will generally have a lower opening with either a tube adapter (203) connected to the distal opening of a hollow tube, or alternatively a directly connected hollow tube (204). This hollow tube (204) will be made of a material (often plastic as well) selected for transporting fluids for human consumption, and will generally be long enough to deliver fluid from the fluid container (while suspended near the patient) to the patient's mouth. At the patient's mouth end, the hollow tube will have a mouthpiece (206) attached to the proximal opening of the hollow tube (204).

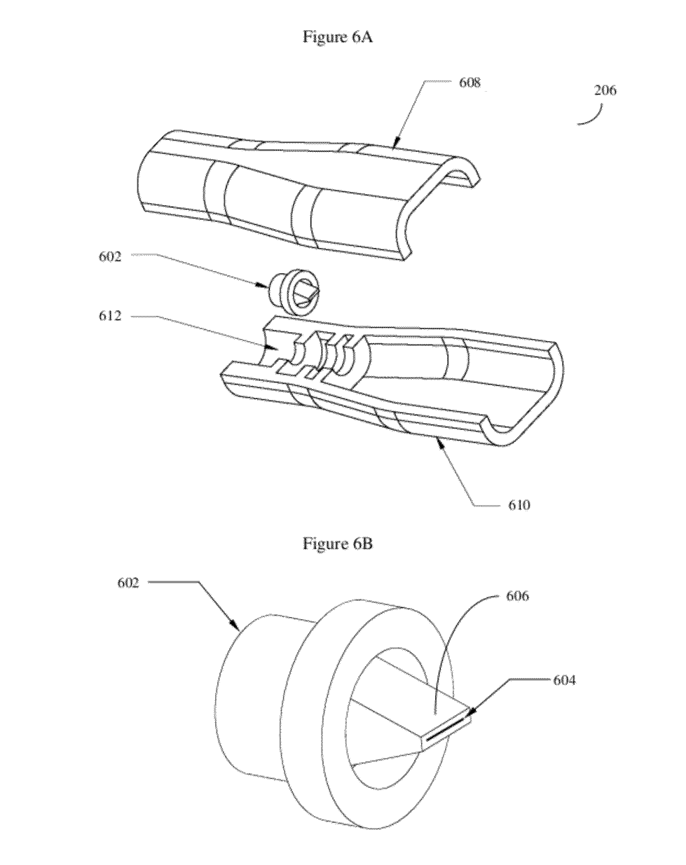

As will be discussed in more detail in FIGS. 6A and 6B, this mouthpiece (206) that connects to the tube (often via a coupling region (612)) will generally be configured with at least a fluid valve (602) comprising at least one narrow slit (604) in an elastic and deformable surrounding material (606). In some embodiments, this fluid valve may be further encased in an outer covering or shell (608), (610) as well. In the absence of patient mouth generated force (e.g. sucking, biting), this deformable material (606) will hold the slit (604) shut, thus preventing fluid flow. However in the presence of patient mouth generated force (e.g. sucking, biting), said deformable material (606) deforms thus causing the slit (604) to enlarge and permitting fluid flow from the container into the patient's mouth. Here, the materials and methods of Fawcett (U.S. Pat. No. 5,085,349) may be used, and these materials and methods are incorporated herein by reference. Other methods (previously discussed) may also be used.

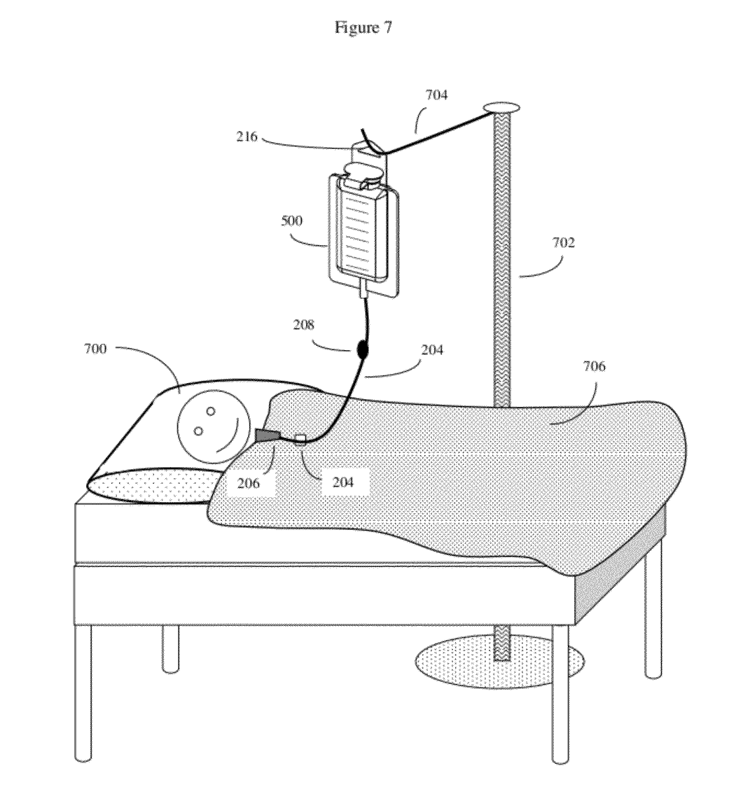

To use the device/method, the container will first be filled with fluid (e.g. water) by opening the press-clamp cap (204), filling the fluid container with fluid, and closing the press-clamp cap. Alternatively before, after, or during this step, the container will be mounted on a support structure (e.g. IV pole) by placing some support member through the fenestrated opening (216). This is shown in more detail in FIG. 7. After this, the tube and mouthpiece can be provided to the patient (e.g. clipped to a convenient location where the patient can easily access) and as needed, the patient can be instructed on what sort of mouth force is best to use the device.

A variety of different types of container designs may be used. In general, containers that have a semi-rigid or rigid back (214), optionally (218) have some advantages in that they provide some structure and support to the container even when it is empty, thus making for more accurate fluid measurements as well as increased ease of handling. At the same time, although all sides of the container may be rigid in some embodiments, often it is useful to have the front of the container (e.g. the side with graduations (212)). as well as the container sides connecting to the optionally semi-rigid or rigid back, be flexible so that they deform outward in response to fluid, and deform inward in response to loss of fluid. This as the container is drained of fluid, the cap (204) can maintain a tight seal, while at the same time a vacuum will not form inside the container (because the container walls will move inward as fluid is depleted), making it uniformly easy for the patient to obtain the fluid, as the fluid levels drop, without any admixture of air or outside contaminants.

In some embodiments, it may be useful to further have a thumb controlled tubing clamp (208) or other secondary fluid shutoff configured to provide an independent shut-off for stopping fluid flow along the hollow tube (204). Additionally, in some embodiments, it may also be useful to have a clip (210) attached to either the tube or the mouthpiece and configured to at least temporarily fix the mouthpiece to a location near the patient's mouth.

In some embodiments, it may be useful to have the top of the container where the cap is made, at least in part, from a relatively rigid material so that the cap has a complementary port to clip on to that will maintain its rigidity and fluid tightness between both full and empty states. Usually it is also useful to make some of the sides of the container, and preferably all of the sides of the container out of a transparent material so that the level of fluid in the container can be easily determined by sight.

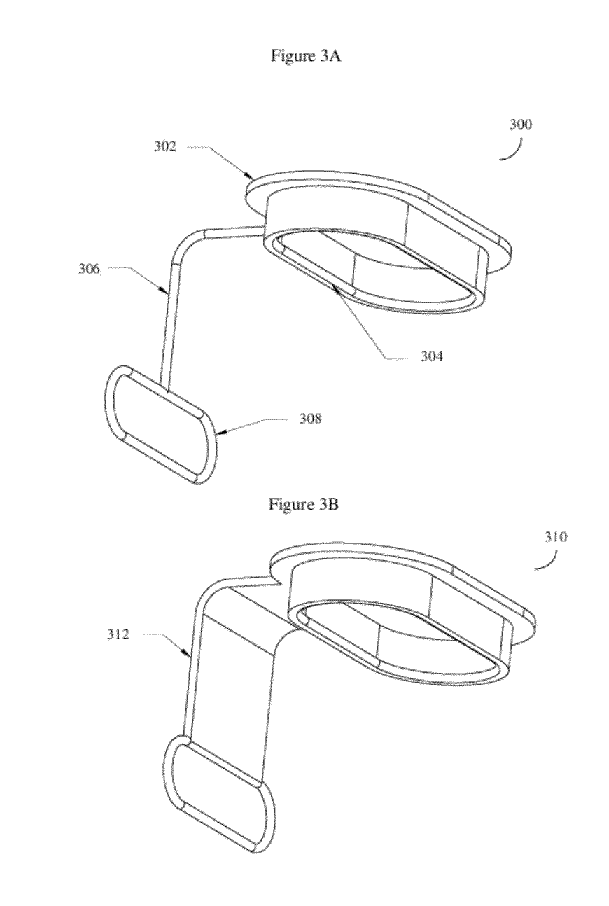

FIG. 3A shows a close up of one embodiment of the devices' snap-on cap assembly (204), here showing one cap design (300). This cap has a flanged edge (302) to provide a good hand grip for opening and closing, an inner ring to snap onto the cap's complementary edge (304), a thin hinged region (306) and a plastic ring (308) to keep the cap adhered to the cap's complementary edge.

FIG. 3B shows a close up of an alternate embodiment of the cap (310) where the hinged region is appreciably wider (312).

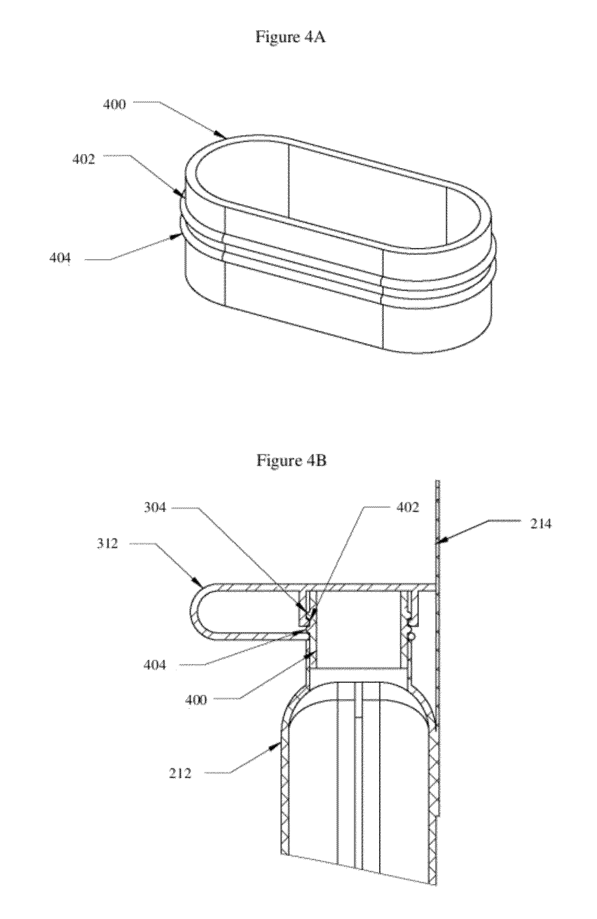

FIG. 4A shows a close up of the complementary edge (400) of the device's snap-on cap. This will usually be made of rigid or semi-rigid material so as to create a good fluid barrier. This complementary edge may optionally have one or more grooves or detents (402), (404) designed to match up and interlock with corresponding grooves or detents in the cap.

FIG. 4B shows a cross sectional view of the snap-on cap (204), (310) in the shut position.

As previously discussed, the back side of the fluid container may be either mounted on, or directly comprise a substantially flat back support (214) with a fenestrated opening (216) extending above the cap (204). This fenestrated opening may be configured to allow the fluid container to be suspended from an IV pole or other support structure, as is shown in FIG. 7.

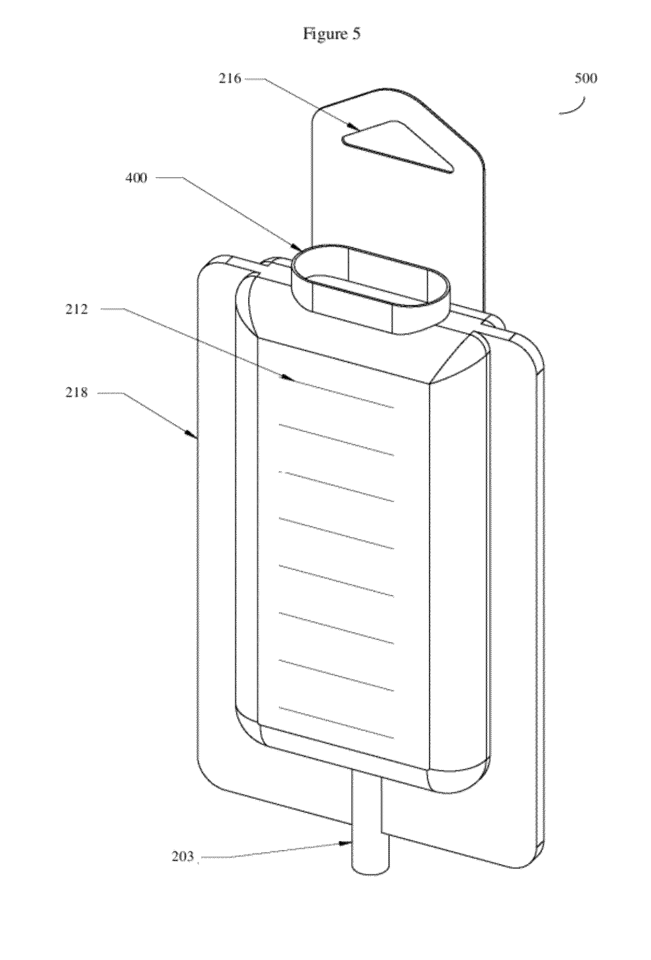

FIG. 5 shows a closer view of the device's fluid reservoir and fenestrated opening. As previously discussed, the fluid container (500) will often have a lower opening with either a tube adapter connected to the distal opening of a hollow tube (203), or a directly connected hollow tube. This hollow tube, previously shown in FIG. 2 as (204), will generally have sufficient length to deliver fluid from the fluid container (500) while the container is suspended near said patient, and deliver this tube to the mouthpiece (206).

FIG. 6A shows an overall view of the device's mouthpiece, and FIG. 6B shows a close up view of the device's valve. As previously discussed, this mouthpiece (206) that connects to the tube (often via a coupling region (612)) will generally be configured with at least a fluid valve (602) comprising at least one narrow slit (604) in an elastic and deformable surrounding material (606). In some embodiments, this fluid valve may be further encased in an outer covering or shell (608), (610) as well. In the absence of patient mouth generated force (e.g. sucking, biting), this deformable material (606) will hold the slit (604) shut, thus preventing fluid flow. However in the presence of patient mouth generated force (e.g. sucking, biting), said deformable material (606) deforms thus causing the slit (604) to enlarge and permitting fluid flow from the container into the patient's mouth. Here, the materials and methods of Fawcett (U.S. Pat. No. 5,085,349) may be used, and these materials and methods are incorporated herein by reference. Other methods (previously discussed) may also be used.

Thus in this particular embodiment, in the absence of patient mouth generated force, the deformable material (606) holds the slit (604) shut, thus preventing fluid flow to the mouth of said patient. However when the patient wants fluid, and communicates this by sucking on the mouthpiece or biting on the mouthpiece, this deformable material deforms thus causing the slit to enlarge and permitting fluid flow to the patent's mouth.

FIG. 7 shows an example of the device in use hydrating a patient (700). In this example, the container (500) is hanging from an IV pole (702) support (704) by way of opening (216). The mouthpiece (206) in this example is clipped to the patient's blanket (706) in a region near the patient's mouth by a clip (210).

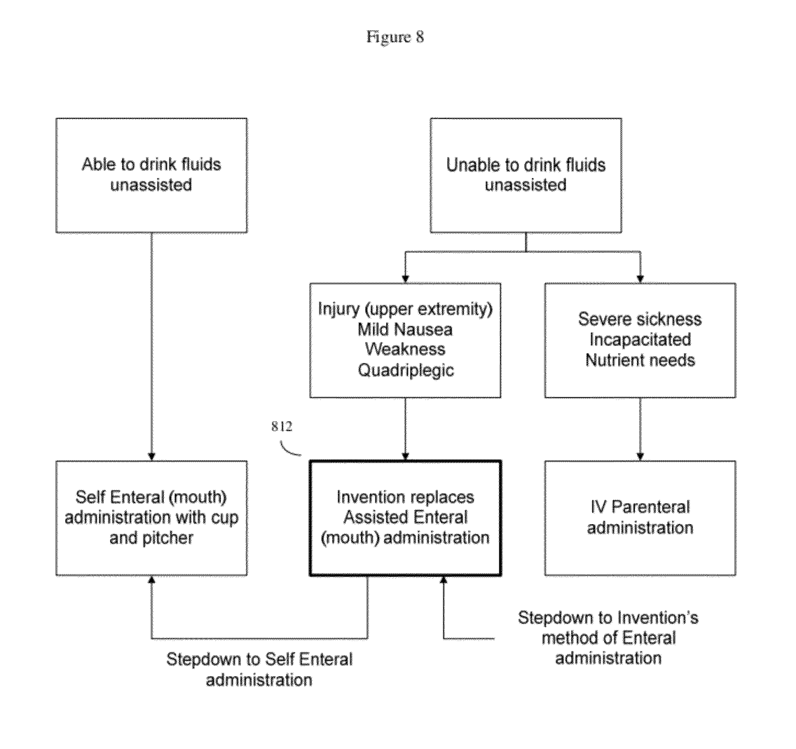

FIG. 8 shows a flow chart of the hydration decision tree and hydration options generally used for patient hydration management, when using the invention. Here the considerations are generally similar to those previously described in FIG. 1, however now with the use of the invention, what was formerly a very labor intensive assisted enteral fluid administration step (112) now is greatly labor reduced because the invention's device and methods are now used in (812). Indeed, the invention reduces the amount of effort required for the patient to self administer fluids to the point where some patients may wish to continue using it even after they regain the ability to use a cup.

Here, the invention's use on a non-obligatory basis (i.e. use by patients capable of self enteral administration) can be as policy and costs dictate. For example the invention's device and methods could be useful in pediatric or geriatric wards where patients can drink with a cup, but may be unacceptably messy while doing so. Thus the invention can reduce clutter and traffic around the patient, help prevent fluid spills, and allow for fluid intake by ambulatory patients.

The device will most commonly be made from one or more plastics. Here the plastics will often be selected from the group of transparent polymers generally recognized as suitable for fluid storage and transport in a medical setting. This can include polyethylene, polypropylene, polyvinyl chloride (PVC), Polyolefin, Thermoplastic elastomers, and the like.

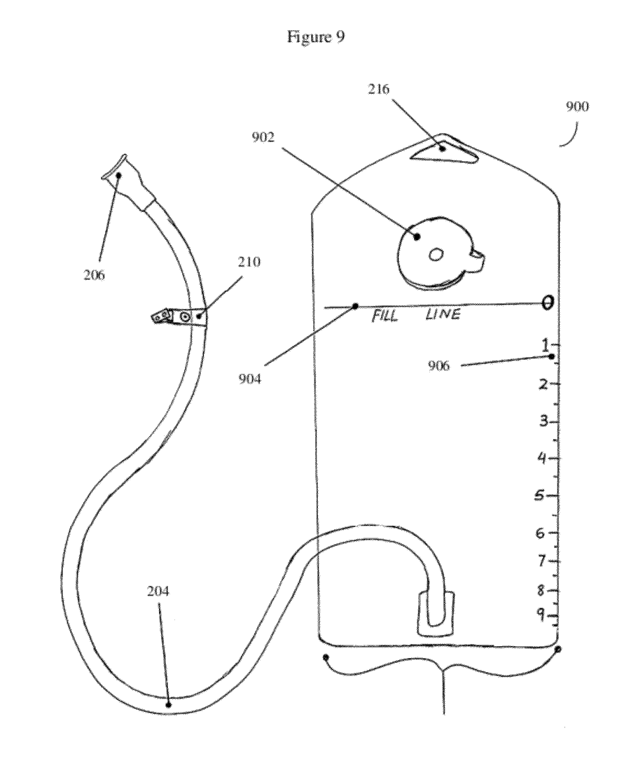

FIG. 9 shows an alternate embodiment of the invention's fluid container. In this embodiment, the entire body of container (900) is made from a flexible and deformable material. The cap (902) is smaller and circular. There are also separate fill line (904) and graduation (e.g. volume) markers (906). The back may optionally be made from a non-deformable material.

1. A method of enterally hydrating a patient, said method comprising:
providing a substantially transparent fluid container comprising a plurality of substantially deformable sides and at least one substantially non-deformable sides;
the top side of said fluid container comprising a substantially non-deformable side with a one-piece rigid or semi-rigid press-clamp cap configured to either open to admit fluids, or snap shut;
the back side of said fluid container mounted on or comprising a substantially flat back support with a fenestrated opening extending above said cap, said fenestrated opening configured to enable said fluid container to be suspended from an IV pole or other support structure;
said fluid container further comprising a lower opening with either a tube adapter connected to the distal opening of a hollow tube, or a directly connected hollow tube, said hollow tube being sufficient length to deliver fluid from said fluid container while suspended near said patient to a hollow tube connected mouthpiece attached to the proximal opening of said hollow tube;
said mouthpiece configured with a valve comprising at least one narrow slit in an elastic and deformable material;
wherein in the absence of patient mouth generated force, said deformable material holds said slit shut, thus preventing fluid flow;
wherein in the presence of patient mouth generated force, said deformable material deforms thus causing said slit to enlarge and permitting fluid flow;
opening said press-clamp cap, filling said fluid container with fluid for said patient; and closing said press-clamp cap;
mounting said container on a support structure using said fenestrated opening;
providing said tube and mouthpiece to said patient, and instructing said patient to apply mouth force when fluid is desired.

2. The method of claim 1, wherein at least one of said container sides contains a fill line and a series of graduated markings and numbers set at various fluid levels so that the quantity of fluid used by said patient may be accurately determined.

3. The method of claim 1, wherein said substantially flat back support with a fenestrated opening extending above said cap is made from a substantially rigid material.

4. The method of claim 1, wherein said valve is configured to open in response to the patient mouth generated force of biting.

5. The method of claim 1, wherein said valve is configured to open in response to the patient mouth generated force of suction.

6. The method of claim 1, wherein said patient is unable to use a cup without assistance.

7. The method of claim 1, wherein said hollow tube further comprises a thumb controlled tubing clamp configured to provide an independent method of stopping fluid flow along said hollow tube.

8. The method of claim 1, wherein said hollow tube proximate said mouthpiece, or said mouthpiece, further comprises a clip configured to fix said mouthpiece to a location near the mouth of said patient.

9. The method of claim 1, wherein said container and hollow tube are formed from a polymer selected from the group of transparent polymers generally recognized as suitable for fluid storage and transport in a medical setting.

10. A device for enterally hydrating a patient, said device comprising:
a substantially transparent fluid container comprising a plurality of substantially deformable sides;
the top side of said fluid container comprising a one-piece rigid or semi-rigid press-clamp cap configured to either open to admit fluids, or snap shut;
the back side of said fluid container mounted on or comprising a back support with a fenestrated opening extending above said cap, said fenestrated opening configured to enable said fluid container to be suspended from an IV pole or other support structure;
said fluid container further comprising a lower opening with either a tube adapter connected to the distal opening of a hollow tube, or a directly connected hollow tube, said hollow tube being sufficient length to deliver fluid from said fluid container while suspended near said patient to a hollow tube connected mouthpiece attached to the proximal opening of said hollow tube;
said mouthpiece configured with a valve comprising at least one narrow slit in an elastic and deformable material;
wherein in the absence of patient mouth generated force, said deformable material holds said slit shut, thus preventing fluid flow to the mouth of said patient;
wherein in the presence of patient mouth generated force, said deformable material deforms thus causing said slit to enlarge and permitting fluid flow to the mouth of said patent.

11. The device of claim 10, wherein at least one of said container sides contains a fill line and a series of graduated markings and numbers set at various fluid levels so that the quantity of fluid used by said patient may be accurately determined.

12. The device of claim 10, wherein said substantially flat back support with a fenestrated opening extending above said cap is made from a substantially rigid material.

13. The device of claim 10, wherein said valve is configured to open in response to the patient mouth generated force of biting.

14. The device of claim 10, wherein said valve is configured to open in response to the patient mouth generated force of suction.

15. The device of claim 10, wherein said patient is unable to use a cup without assistance.

16. The device of claim 10, wherein said hollow tube further comprises a thumb controlled tubing clamp configured to provide an independent shut-off for stopping fluid flow along said hollow tube.

17. The device of claim 10, wherein said hollow tube proximate said mouthpiece, or said mouthpiece, further comprises a clip configured to fix said mouthpiece to a location near the mouth of said patient.

18. The device of claim 10, wherein said container and hollow tube are formed from a polymer selected from the group of transparent polymers generally recognized as suitable for fluid storage and transport in a medical setting.

19. The device of claim 10, wherein said device additionally comprises at least one substantially non-deformable side;
said top side of said fluid container further comprises a substantially non-deformable side; and
said back side of said fluid container further comprises a substantially flat back support.

20. A method of enterally hydrating a patient, said method comprising:
providing a substantially transparent fluid container comprising a plurality of substantially deformable sides;
the top side of said fluid container comprising a one-piece rigid or semi-rigid press-clamp cap configured to either open to admit fluids, or snap shut;
the back side of said fluid container mounted on or comprising a back support with a fenestrated opening extending above said cap, said fenestrated opening configured to enable said fluid container to be suspended from an IV pole or other support structure;
said fluid container further comprising a lower opening with either a tube adapter connected to the distal opening of a hollow tube, or a directly connected hollow tube, said hollow tube being sufficient length to deliver fluid from said fluid container while suspended near said patient to a hollow tube connected mouthpiece attached to the proximal opening of said hollow tube;
said mouthpiece configured with a valve comprising at least one narrow slit in an elastic and deformable material;
wherein in the absence of patient mouth generated force, said deformable material holds said slit shut, thus preventing fluid flow;
wherein in the presence of patient mouth generated force, said deformable material deforms thus causing said slit to enlarge and permitting fluid flow;
opening said press-clamp cap, filling said fluid container with fluid for said patient; and closing said press-clamp cap;
mounting said container on a support structure using said fenestrated opening;
providing said tube and mouthpiece to said patient, and instructing said patient to apply mouth force when fluid is desired.
